(19) United States
(12) Patent Application Publication
(10) Pub. No.: US 2022/0290190 A1
(43) Pub. Date: Sep. 15, 2022

(54) METHODS FOR EXTRACTING OILS FROM OIL EMULSIONS USING CHEMICAL TREATMENT

(71) Applicant: CHEMTREAT, INC., Glen Allen, VA (US)

(72) Inventors: William HENDERSON, Ashland, VA (US); Robert BEDINGER, Glen Allen, VA (US); Matthew GITTINGS, Glen Allen, VA (US); Joshua HOFFMAN, Glen Allen, VA (US)

(73) Assignee: CHEMTREAT, INC., Glen Allen, VA (US)

(21) Appl. No.: 17/692,799

(22) Filed: Mar. 11, 2022

Related U.S. Application Data
(60) Provisional application No. 63/160,394, filed on Mar. 12, 2021.

(51) Int. Cl. C12P 7/06; B01D 17/04; C10L 1/02; C11B 1/10

(52) U.S. Cl. CPC C12P 7/06; B01D 17/047; C10L 1/02; C11B 1/10; C10L 2200/0484; C10L 2290/58

(57) ABSTRACT

A method for applying a chemical treatment to a process stream in an ethanol production/corn oil extraction system. The method includes applying the chemical treatment to the process stream before or during fermentation of an emulsified oil precursor product in the process stream to demulsify the emulsified oil precursor product, and extracting the demulsified oil from the emulsified oil precursor product.

### TECHNICAL FIELD

This application relates to methods for producing demulsified oil and extracting oils from oil emulsions using chemical treatment.

### BACKGROUND

Demulsified oils are used for many purposes in foods and cosmetics. One common demulsified oil is corn oil. Corn oil exists within the germ of the kernel and is a by-product of the corn oil milling process. Most corn that is harvested is used as feed but the proportion of the corn that is milled is increasing because of bioethanol production. During the wet milling process, the germ is isolated from the starch using cyclone separators, washed, and dried.

In ethanol production, after ethanol is stripped from fermentation fluids, ethanol production facilities are typically left with whole stillage. The whole stillage is separated into corn solids and centrate. Some of the centrate is recycled and goes back into fermentation, while some goes to evaporators as thin stillage to be concentrated into syrup. Corn oil is then extracted from the syrup.

Corn oil is sold as a value added product and acts as a revenue source for ethanol production/corn oil extraction facilities. Any corn oil that is not recovered is lost revenue.

Therefore, improvements in oil extraction from this process result in added revenue for the facilities. In order to increase corn oil yield, chemical companies have been known to supply an additive or separation aid to remove the available oil in the syrup. Separation aids for corn oil are conventionally applied to the syrup after the thin stillage has been thickened in evaporators in order to separate corn oil. However, these processes lack efficiency and can be expensive. Moreover, attempts to modify other points of the corn oil production process have been met with significant resistance due to the negative impact on ethanol production. As a result, there is a substantial need for better methods for producing corn oil with higher yields, and without negatively impacting ethanol yield.

### SUMMARY

The disclosed methods and systems solve these and other problems by applying chemical treatment at previously untried stages in a process stream of an oil extraction system, e.g., an ethanol production/corn oil extraction system. In one aspect, for example, the methods can release the oil earlier in the ethanol/corn oil production pathway to bring more oil into the syrup. As a result, it was discovered that increased oil yields in oil production facilities can be realized, which increases the profitability of the facilities by hundreds of thousands of dollars or more per year. According to some aspects of this disclosure, chemical treatment is applied prior to or during fermentation, which increases the available free oil in the back-end syrup.

In an embodiment, there is provided a method for applying a chemical treatment to a process stream in an oil extraction system. The method includes applying the chemical treatment including a demulsifier to the process stream before or during fermentation of an emulsified oil precursor product in the process stream to demulsify the emulsified oil precursor product, and extracting the demulsified oil from the emulsified oil precursor product

### BRIEF DESCRIPTION OF THE DRAWINGS

FIG. 1 is a schematic diagram of an ethanol production/corn oil extraction process according to an embodiment.

FIG. 2 illustrates corn oil production results of a data capture system in an ethanol production/corn oil extraction facility according to disclosed examples.

### DETAILED DESCRIPTION

Disclosed embodiments employ a chemical treatment that is added to a process stream in an ethanol production/corn oil extraction system to demulsify a precursor emulsified oil product, and extract the demulsified oil from the precursor emulsified oil product. For purposes of this disclosure, the oil extraction process will be described with respect to an ethanol production/corn oil extraction system and the oil will be described with respect to corn oil. However, it will be recognized that the system and oil can be any suitable system or oil including, but not limited to, vegetable oil, animal oil, animal fat, petroleum oil, mixtures thereof, and the like, and systems for processing the same.

In corn oil production, care must be taken to choose a chemical treatment that does not upset the fermentation process, which would decrease ethanol yield. Accordingly, ethanol producers are hesitant to add chemistries to the fermentation process. In contrast to this approach, it was discovered by the inventors that, by adding the chemical treatment to a fluid used in the fermentation process or before corn solids separation, the yield of corn oil is increased significantly. The increased production of corn oil is used as a revenue source for the ethanol/corn oil production facility.

The Chemical Treatment

In embodiments, the chemical treatment may include any suitable oil surfactant or demulsifier. For example, the demulsifier may be an alkoxylated chemistry. The term “alkoxylated” is used herein as an adjective that describes a material as having been a reactant in a chemical reaction during which alkoxy groups were added to the material. An alkoxy functional group (or alkyl oxide) is an alkyl group singular bonded to oxygen. The simplest alkoxy groups are methoxy (CH₃O—), ethoxy (CH₃CH₂O—), propoxy (CH₃CH₂CH₂O—), and isopropoxy. The general form of an ethoxylation reaction is given as:

ROH+n C₂H₄O→R(OC₂H₄)ₙOH.

A polyoxyethylene group would have repeat units of oxyethylene (—OC₂H₄—) and is formed in an ethoxylation reaction using ethyl oxide.

In embodiments, the emulsifier may be a PEG-ylated sorbitan esterified with fatty acids, commonly known as polysorbates. Polysorbates are non-ionic surfactants and demulsifiers used in foods and cosmetics and are derived from polyethoxylated sorbitan and oleic acid. Non-limiting example polysorbates include polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), and polysorbate 80 (polyoxyethylene (20) sorbitan monooleate), where the number following the polyoxyethylene part refers to the total number of oxyethylene (—CH2CH2O—) groups found in the molecule. The demulsifier may include from 15 to 200 oxyethylene groups, from 50 to 150 oxyethylene groups, or from 50 to 100 oxyethylene groups. In preferred embodiments, polysorbate 80 is used as the demulsifier in the chemical treatment. Polysorbate 80 is available as Tween® 80 which is (polyoxyethylene (20) sorbitan monooleate) and is shown below:

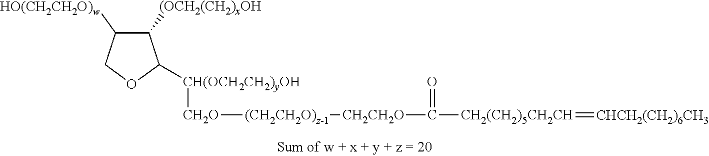

The oil surfactant or demulsifier may be added to a process stream in the ethanol production/corn oil extraction process in the form of a composition or liquid treatment solution alone (e.g., in pure form) or along with other constituents. Other constituents in the chemical treatment may include, but are not limited to, silicon containing particles such as, for example, from silica, talc, clay, diatomaceous earth, and mixtures thereof, fatty acids such as, for example, capric acid, lauric acid, myristic acid, palmitic acid, linoleic acid, stearic acid, linolenic acid, and oleic acid, and glycols such as, for example, polyethylene glycol, a polypropylene glycol, a polyethylene glycol derivative, and mixtures thereof.

The demulsifier of the chemical treatment may be present in the composition or solution in any suitable amount. For example, it may be present at the stage it is applied to the production stage in a range of 5% to 100 wt%, 10% to 95 wt%, 20% to 90 wt%, 25% to 85 wt%, 30% to 70 wt%, 40% to 60 wt%, or 45% to 50 wt%, where all weight percentages are percent by weight of the total composition or solution. The demulsifier may be present in the process stream to which the chemical treatment is added in a range of 0.01 to 10,000 ppm, 0.1 to 5,000 ppm, 1 to 2,500 ppm, 1 to 1,000 ppm, or 1 to 200 ppm.

Application of the Treatment Chemistry

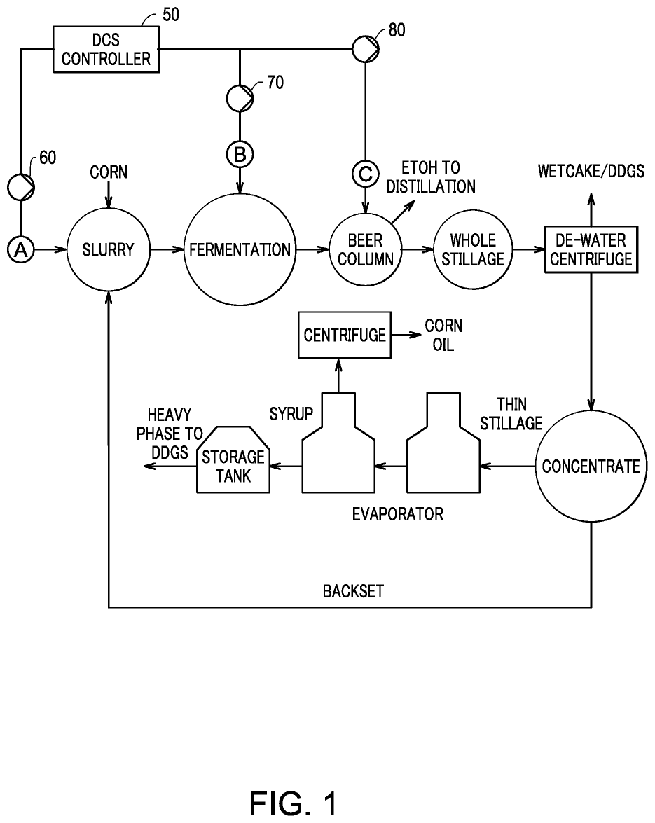

With reference to FIG. 1, in the ethanol production/corn oil extraction process according to embodiments, the corn kernel gets pulverized in hammer mills in a dry grind process to make the particles smaller and increase surface area for enzymatic breakdown to starch, which later gets converted by yeast to sugar. This mechanical and chemical change to the corn kernel binds the oil to the solids. The pulverized corn is then mixed into a slurry and the slurry is fermented. Ethanol is removed from fermented corn slurry in a beer column during a distillation process. After distillation to remove the ethanol, the spent corn slurry or whole stillage is processed through a de-water centrifuge to separate the solids (wet cake) from the liquids or centrate, i.e., concentrate. The wet cake discharged from the centrifuge is conveyed either to the wet cake storage pad for sale locally as wet livestock feed and/or to the distiller's dry grain and solubles (DDGS) dryer to produce a dry livestock feed.

The concentrate flow splits to become one stream which goes to slurry via a backset and the other stream is considered to be thin stillage. Thin stillage discharged from the centrifuge is processed through a closed loop evaporator system to remove water. Water vapor from the evaporator is captured, condensed, and collected for re-use in the process. The remaining concentrated liquid discharged from the evaporator system is referred to as “syrup” which contains carbohydrates, fats (corn oil), sugars, and proteins. The syrup is pumped to a storage tank and is then applied to the wet cake discharged from the centrifuge or added to the distiller's grains run through the dryers. Heated thin stillage from the pressure tank is fed to a stacked disc centrifuge to separate and remove corn oil. The centrifuge separates corn oil from sludge and defatted thin stillage. Upon exiting the centrifuge, the corn oil is directed to the oil recovery tank.

In embodiments, the chemical treatment is applied to the process stream in the ethanol production/corn oil extraction process before or during fermentation, and is present at least prior to corn solids separation via the centrifuge to separate the solids from the liquids or centrate. For example, the chemical treatment can be added to the slurry or slurry precursor components (point A), directly to the fermentation tank (point B), or the beer column (point C), as seen in FIG. 1. Without intending to be bound by theory, it is believed that this chemical treatment feed-points separate corn oil from oil coated solids and/or is able to penetrate deeper into solids to remove oil during the fermentation process. Additionally, the treatment may affect the fermentation fluids in the process stream in a way prior to fermentation that destabilizes the oil in water emulsion and allows for increased separation post-fermentation. It is further believed that when the corn is enzymatically degraded, the disclosed methods “neutralize” the charge which has the affinity towards binding the oil.

The primary demulsifier according to embodiments may be non-ionic. As oil is enzymatically broken down during fermentation, the oil becomes stabilized in the emulsion.

Disclosed methods prevent this stabilization, thereby inhibiting emulsion. By treating the process stream earlier along in the process, i.e., before or during fermentation, disclosed embodiments provide for surprising advantages in terms of increased recovery of extracted oil. Conventional techniques that provide treatment after emulsion depend on much higher concentrations of demulsifiers. It is well known in the art of water treatment that the time necessary to separate emulsions is based on particle size, viscosity, and time (i.e., Stokes Law). Disclosed embodiments provide for creating larger particle sizes up front, i.e., before or during fermentation. The larger particles in solution attract other particles according to Stokes Law and thus make separation or demulsification easier than was conventionally possible.

Importantly, the disclosed embodiments allow for chemical treatment to be applied to the process stream in the ethanol production/corn oil extraction system without reducing the ethanol produced from the beer column. In this regard, the anaerobic process of fermentation occurs independently and materially unaffected by the corn oil separation process described above.

Disclosed methods may be employed while an ethanol production/corn oil extraction system implementing the ethanol production/corn oil extraction process is online or offline. As used herein, the term “online” refers to application of the chemical treatment while the system is operational and fermentation fluids are processed through equipment as opposed to being “offline” when operation ceases.

Treatment Chemistry Feed Automation and Control

Using the methods described herein, chemical treatments are able to be controlled, i.e., adjusted and optimized, while the system is online, thereby increasing overall efficiency and reducing costs. A feed system feeds the chemical treatments into the process stream upstream of the fermenter, as illustrated in FIG. 1. According to embodiments, chemical treatment feed rates can be precisely and accurately controlled.

The dosage and rate control plan for the application of the chemical treatments will be dependent upon the specific contents of the chemical treatments, the control plan and system operating conditions. According to the disclosed methods, the dosage amounts and rates can be developed for each chemical treatment applied, to thereby allow for the change in dosage amounts and rates.

A control feed architecture according to embodiments may include at least one circulation pump for circulating the fermentation fluid flow through the system and at least one chemical treatment solution pump for feeding the chemical treatment into the process stream of the ethanol production/corn oil extraction process. Multiple circulation pumps and/or chemical treatment pumps 60, 70, and 80 may be provided in order to accommodate required volumes of fluid running through the system. A distributed control system (DCS) controller 50 may control the operation of these pumps based on demand driven by various system parameters, e.g., operational load. The DCS controller 50 controls the overall operation of the facility and is where the plant instrumentation sends its data, and may include, for example, tens of thousands of data points. The architecture may further include a data capture panel for receiving operations input and providing the DCS controller 50 with the appropriate instructions for controlling the operation of the pumps.

The proper chemical treatment dosage and rate can be adjusted real-time using recorded parameters. Once the dosage amounts and rates are calculated, these schemes may be stored in the storage for historical purposes. The schemes are then accessed by the DCS controller 50 when appropriate and applied to the ethanol production/corn oil extraction system via the control feed architecture. The control feed architecture adjusts the amount and/or rate of the chemical treatment by, for example, calculating the ml/min set point to control and adjust the various chemical feed pumps to control flow from the chemical treatment source to the ethanol production/corn oil extraction system. The dosage schemes for each specific chemical treatment are optimized in this manner.

Additionally, the programmable logic behind the dosing and application rate can be refined in the field in response to real-time real-world conditions and performance at the site. And adjustments to dosing and application rate can be made virtually instantaneously. As a result, the disclosed embodiments will provide real-time and more effective oil control management compared to conventional processes by improving the overall reliability, efficiency, and economic productivity of the ethanol production/corn oil extraction system.

Embodiments may further include machine learning algorithms implemented on the disclosed controllers for executing the disclosed functions in a predictive manner. For example, the machine learning algorithms may be used to establish historical patterns to predict future feed needs based on any one or more parameters that may include. Outputs of the predictive logic controllers may be connected to external reporting and analysis sites such as an inventory control device.

The programmatic tools used in developing the disclosed machine learning algorithms are not particularly limited and may include, but are not limited to, open source tools, rule engines such as Hadoop®, programming languages including SAS®, SQL, R and Python and various relational database architectures.

Each of the disclosed controllers may be a specialized computer(s) or processing system(s) that may implement machine learning algorithms according to disclosed embodiments. The computer system is only one example of a suitable processing system and is not intended to suggest any limitation as to the scope of use or functionality of embodiments of the methodology described herein. The processing system shown may be operational with numerous other general purpose or special purpose computing system environments or configurations. Examples of well-known computing systems, environments, and/or configurations that may be suitable for use with the disclosed embodiments may include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

The computer system may be described in the general context of computer system executable instructions, such as program modules, being executed by a computer system. Generally, program modules may include routines, programs, objects, components, logic, data structures, and so on that perform particular tasks or implement particular abstract data types. The computer system may be practiced in distributed cloud computing environments where tasks are performed by remote processing devices that are linked through a communications network. In a distributed cloud computing environment, program modules may be located in both local and remote computer system storage media including memory storage devices.

The components of computer system may include, but are not limited to, one or more processors or processing units, a system memory, and a bus that couples various system components including system memory to processor. The processor may include a module that performs the methods described herein. The module may be programmed into the integrated circuits of the processor, or loaded from memory, storage device, or network or combinations thereof.

### EXAMPLES

The following examples were conducted in ethanol production/corn oil extraction systems at three sites. The sites cleaned in place their oil centrifuges. Oil accumulates in the evaporator during the cleaning in place. When the centrifuge gets put online, oil production will temporarily increase to account for the accumulation of oil in the evaporator.

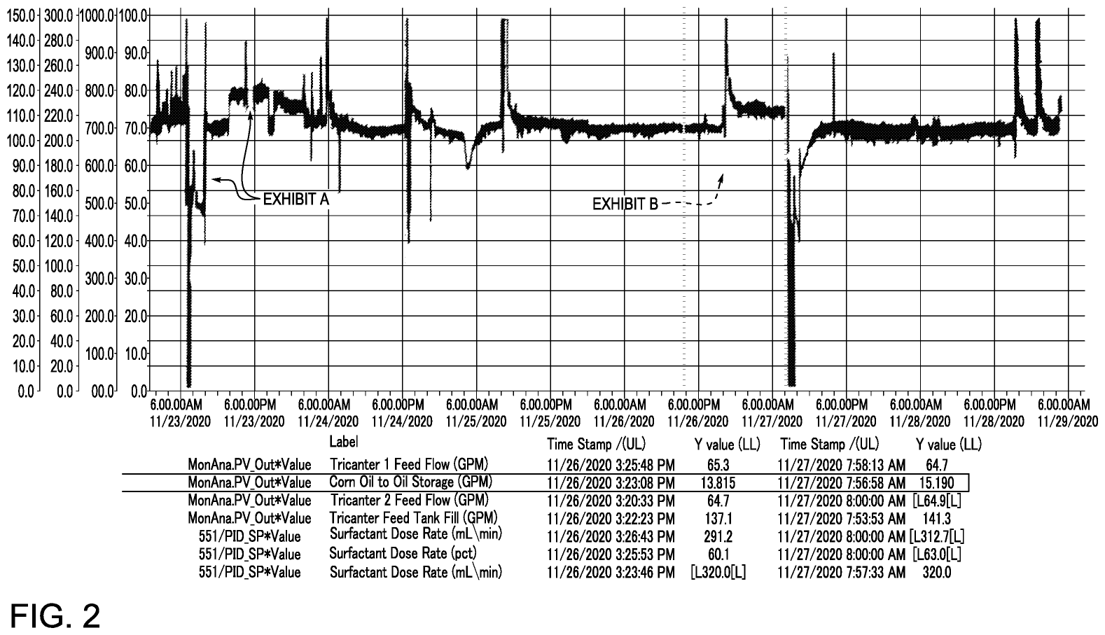

Results indicated that corn oil production was increased by 3.5% (Site I), 6% (Site II), and 10% (Site III) compared to like reference processes where the disclosed chemical treatment was not applied. Some results from Site III are illustrated in FIG. 2. As seen in FIG. 2, Site III averaged about 14 gpm of corn oil (Exhibit A). The two red lines of Exhibit B in FIG. 2 illustrate the period when it was expected that the disclosed chemical treatment completes its journey through the oil centrifuge. For a ten hour period of application of the disclosed chemical treatment, oil production increased from 13.8 to 15.19 gpm of oil. Ethanol yield was unaffected compared to like reference processes where the disclosed chemical treatment was not applied. This increase suggests that the disclosed methods provide clear advantages over conventional treatments and application methods in terms of corn oil production while not negatively impacting ethanol yield.

It will be appreciated that the above-disclosed features and functions, or alternatives thereof, may be desirably combined into different methods and systems. Also, various alternatives, modifications, variations or improvements may be subsequently made by those skilled in the art, and are also intended to be encompassed by the disclosed embodiments. As such, various changes may be made without departing from the spirit and scope of this disclosure.

What is claimed is:

1. A method for applying a chemical treatment to a process stream in an oil extraction system, the method comprising:
applying the chemical treatment including a demulsifier to the process stream before or during fermentation of an emulsified oil precursor product in the process stream to demulsify the emulsified oil precursor product; and
extracting the demulsified oil from the emulsified oil precursor product.

2. The method for applying a chemical treatment to a process stream in an oil extraction system according to claim 1, wherein the chemical treatment is applied to the process stream during fermentation of the emulsified oil precursor product in the process stream.

3. The method for applying a chemical treatment to a process stream in an oil extraction system according to claim 1, wherein the chemical treatment is applied to the process stream before fermentation of the emulsified oil precursor product in the process stream.

4. The method for applying a chemical treatment to a process stream in an oil extraction system according to claim 1, wherein the oil extraction system is part of an ethanol production system.

5. The method for applying a chemical treatment to a process stream in an oil extraction system according to claim 1, wherein the emulsified oil precursor product is a corn slurry and the demulsified oil is corn oil.

6. The method for applying a chemical treatment to a process stream in an oil extraction system according to claim 1, wherein the demulsifier is a non-ionic demulsifier.

7. The method for applying a chemical treatment to a process stream in an oil extraction system according to claim 1, wherein the demulsifier is present in the chemical treatment at a concentration in a range of 5% to 100 wt%.

8. The method for applying a chemical treatment to a process stream in an oil extraction system according to claim 1, wherein the demulsifier is present in the chemical treatment at a concentration in a range of 30% to 70 wt%.

9. The method for applying a chemical treatment to a process stream in an oil extraction system according to claim 1, wherein the demulsifier is present in the chemical treatment at a concentration in a range of 40% to 60 wt%.

10. The method for applying a chemical treatment to a process stream in an oil extraction system according to claim 1, wherein the demulsifier is present in the process stream at a concentration in a range of 0.01 to 10,000 ppm.

11. The method for applying a chemical treatment to a process stream in an oil extraction system according to claim 1, wherein the demulsifier is present in the process stream at a concentration in a range of 1 to 2,500 ppm.

12. The method for applying a chemical treatment to a process stream in an oil extraction system according to claim 1, wherein the demulsifier is present in the process stream at a concentration in a range of 1 to 200 ppm.

13. The method for applying a chemical treatment to a process stream in an oil extraction system according to claim 1, wherein the demulsifier includes an alkoxy group.

14. The method for applying a chemical treatment to a process stream in an oil extraction system according to claim 1, wherein the demulsifier is at least one polysorbate selected from the group consisting of polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), and polysorbate 80 (polyoxyethylene (20) sorbitan monooleate).

15. The method for applying a chemical treatment to a process stream in an oil extraction system according to claim 14, wherein the non-ionic emulsifier is polysorbate 80.

16. The method for applying a chemical treatment to a process stream in an oil extraction system according to claim 1, wherein the chemical treatment further includes at least one constituent selected from the group consisting of silica, talc, clay, diatomaceous earth, capric acid, lauric acid, myristic acid, palmitic acid, linoleic acid, stearic acid, linolenic acid, oleic acid, polyethylene glycol, polypropylene glycol, and a polyethylene glycol derivative.

17. The method for applying a chemical treatment to a process stream in an oil extraction system according to claim 1, further comprising:
determining at least one of a dosage and a rate of application of the chemical treatment; and
controlling application of the chemical treatment to the process stream based on the determined at least one of the dosage and the rate of application.

18. The method for applying a chemical treatment to a process stream in an oil extraction system according to claim 1, wherein the oil extraction system is online during the applying step.

19. The method for applying a chemical treatment to a process stream in an oil extraction system according to claim 1, wherein the oil extraction system is part of an ethanol production system, and
3.5% or more of the demulsified oil is extracted from the emulsified oil precursor product compared to a like reference process where the chemical treatment was not applied.

20. The method for applying a chemical treatment to a process stream in an oil extraction system according to claim 1, wherein the oil extraction system is part of an ethanol production system, and
an amount of ethanol produced during the process was not reduced compared to a like reference process where the chemical treatment was not applied.
